(19) United States
(12) Patent Application Publication
(10) Pub. No.: US 2025/0375244 A1
(43) Pub. Date: Dec. 11, 2025

(54) ABLATION CATHETERS WITH EXPANDABLE ELEMENTS AND BIPOLAR ELECTRODES TO TREAT VARICOSE VEINS

(71) Applicant: Boston Scientific Medical Device Limited, Ballybrit (IE)

(72) Inventors: Yinghua Wang, Shanghai (CN); Mengxiang Luo, Shanghai (CN); Mingfeng Frank Xie, Shanghai (CN); Cheng Zhang, Shanghai (CN); Longsheng Cai, Shanghai (CN); Linshanzi Pam, Shanghai (CN)

(21) Appl. No.: 18/881,705

(22) PCT Filed: Jul. 13, 2023
(86) PCT No.: PCT/EP2023/069501

§ 371 Date: Jan. 6, 2025

(30) Foreign Application Priority Data
Jul. 14, 2022 (CN) 202210833124.2

(51) Int. Cl. A61B 18/14; A61B 18/00; A61B 18/12

(52) U.S. Cl. CPC A61B 18/1492; A61B 18/1206; A61B 2018/0016; A61B 2018/0022; A61B 2018/00404; A61B 2018/00577; A61B 2018/00875; A61B 2018/00904; A61B 2018/126; A61B 2018/1467; A61B 2018/1497

(57) ABSTRACT

At least some embodiments of the present disclosure are directed to a catheter for use in varicose vein treatment including a handle, an elongated shaft connected to the handle, and a heating element disposed near the distal end of the shaft. In some embodiments, the heating element includes an inflatable balloon having a proximal end and an opposite distal end and defining a longitudinal dimension therebetween, and a plurality of electrode sets including elongated electrodes extending along a majority of the longitudinal dimension of the balloon, and the electrodes of each electrode set are configured to form an anode-cathode pair for bipolar delivery of radiofrequency ablative energy to target tissue.

### TECHNICAL FIELD

The present disclosure pertains to medical devices, systems, and methods for providing a therapeutic heat treatment. More particularly, the present disclosure pertains to medical devices, systems and methods for providing therapeutic heat treatments to venous diseases.

### BACKGROUND

Therapeutic heat treatment can be used to treat a wide variety of medical conditions such as tumors, fungal growth, etc. Heat treatments can be used for treating medical conditions alongside other therapeutic approaches or as a standalone therapy. Heat treatment provides localized heating and thus does not cause any cumulative toxicity in contrast to other treatment methods such as drug-based therapy, for example.

One exemplary clinical application of therapeutic heat treatment is in the treatment of chronic venous diseases such as varicose veins, which may become enlarged and/or tortuous due to one or more pathological conditions. Application of sufficient thermal energy via an intravascular device can treat varicose veins by constricting or occluding the target veins.

There is a continuing need for improved devices and methods to provide focused, controlled thermal energy for thermally treating chronic venous conditions such as varicose veins while minimizing or eliminating effects on surrounding healthy tissue.

### SUMMARY

In Example 1, a device for treating varicose vein includes a catheter including an elongated shaft having a proximal end and a distal end, the shaft being sized and configured such that the distal end can be inserted into a target blood vessel; and a heating element disposed near the distal end of the elongated shaft. The heating element may include an inflatable balloon having a proximal end and an opposite distal end and defining a longitudinal dimension therebetween; and a plurality of electrode sets disposed circumferentially about the balloon, wherein each electrode set comprises first and second elongated electrodes extending along a majority of the longitudinal dimension of the balloon, wherein the electrodes of each electrode set are configured to form an anode-cathode pair for bipolar delivery of radiofrequency ablative energy to target tissue of the target blood vessels.

In Example 2, the device of Example 1, wherein the inflatable balloon has a length greater than three (3) centimeters.

In Example 3, the device of Example 2, wherein the inflatable balloon has a length smaller than ten (10) centimeters.

In Example 4, the device of Example 1, wherein the inflatable balloon has a diameter greater than five (5) millimeters when inflated.

In Example 5, the device of Example 1, wherein the inflatable balloon has a diameter greater than twelve (12) millimeters when inflated.

In Example 6, the device of Example 1, wherein the inflatable balloon has a diameter greater than a diameter of the target blood vessel when inflated.

In Example 7, the device of Example 1, wherein the inflatable balloon has a length and a diameter, wherein the length is at least two times of the diameter when inflated.

In Example 8, the device of Example 1, wherein at least one electrode in the plurality of electrode sets comprises a flexible circuit.

In Example 9, the device of Example 1, wherein a distance between an anode-cathode pair is smaller than a distance between two adjacent electrode sets.

In Example 10, the device of Example 9, the distance between two adjacent electrode sets is at least two (2) times of the distance between the anode-cathode pair.

In Example 11, a system for treating varicose vein includes the device of any of Examples 1-10; an energy generator connected to the catheter and configured to generate an electric signal; and a controller operatively connected to the energy generator to control the generation of the electric signal.

In Example 12, the system of Example 11, wherein the plurality of electrode sets are operatively coupled to the energy generator.

In Example 13, the system of Example 11, wherein the inflatable balloon are inflated to a first diameter at a first operating mode and the inflatable balloon is inflated to a second diameter at a second operating mode, wherein the first diameter is different from the second diameter.

In Example 14, the system of Example 13, wherein the inflatable balloon is inflated to a diameter such that an expandable membrane of the inflatable balloon is pressed against a wall of the target blood vessel.

In Example 15, the system of Example 14, wherein the controller is configured to receive a measured impedance between the plurality of electrode sets to determine if the inflatable balloon is contacting the wall of the target blood vessel.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic illustration of an exemplary ablation device for treating chronic venous diseases, e.g., varicose veins, according to an embodiment of the present disclosure.

FIG. 2A is a schematic illustration of an exemplary ablation catheter including a connector for treating chronic venous diseases, e.g., varicose veins, according to an embodiment of the present disclosure.

FIG. 2B is a schematic cross-sectional view of the connector of the exemplary ablation catheter of FIG. 2A, according to embodiments of the present disclosure.

FIG. 2C is a schematic cross-sectional view of the handle of the exemplary ablation catheter of FIG. 2A, according to embodiments of the present disclosure.

FIG. 3 is a schematic partial blown-up view of the distal end portion of an ablation catheter, according to embodiments of the present disclosure.

FIGS. 4A and 4B are schematic illustrations of a portion of an ablation catheter for use in a target blood vessel in a patient for treatment of varicose veins, according to embodiments of the present disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

The following detailed description is exemplary in nature and is not intended to limit the scope, applicability, or configuration of the invention in any way. Rather, the following description provides some practical illustrations for implementing exemplary embodiments of the present invention. Examples of constructions, materials, and/or dimensions are provided for selected elements. Those skilled in the art will recognize that many of the noted examples have a variety of suitable alternatives.

Therapeutic heat treatment can be used to treat a wide variety of medical conditions including chronic venous diseases such as varicose veins, which may become enlarged and/or tortuous due to one or more pathological conditions. Application of sufficient thermal energy via an intravascular device can treat varicose veins by constricting or occluding the target veins.

An exemplary catheter for use in varicose vein treatment may include a handle, an elongated shaft connected to the handle, and a heating element disposed near the distal end of the shaft. In some embodiments, the heating element may receive currents (e.g., alternating currents, direct currents) delivered by an energy generator to generate and deliver thermal ablative energy. In certain embodiments, the heating element may receive electrical signals (e.g., radiofrequency alternating currents) generated by an energy generator to generate and deliver radiofrequency ablative energy.

As mentioned above, there is a continuing need for improved devices and methods to provide focused, controlled thermal energy for thermally treating chronic venous conditions such as varicose veins while minimizing or eliminating effects on surrounding healthy tissue. For example, the diameter of the varicose vein being treated may vary depending on the patient, or the location of treatment (e.g., the Greater Saphenous Vein may range in diameter from about 2.5 mm to about 14.0 mm at the femoral junction, from about 1.5 mm to about 12.0 mm in the thigh, and from about 1.0 mm to about 8.0 mm in the calf. The Lesser Saphenous Vein may range from about 1.5 mm to about 3.0 mm). Heat treatment delivered may not be efficient or effective if a same sized catheter is used for treating veins with different diameters. In certain situations, it may be desired for the heating element to completely occlude the target vein during treatment. In addition, increased flexibility is desired on the catheter used to treat target blood vessel to minimize potential undesirable harm to vessel walls during treatment.

Some embodiments of the present disclosure describe a catheter with an elongated shaft and a heating element disposed near the distal end of the elongated shaft. In some embodiments, the heating element includes an inflatable balloon having a proximal end and an opposite distal end and defining a longitudinal dimension therebetween, and a plurality of electrode sets circumferentially spaced about the inflatable balloon and operatively coupled to the energy generator. In some embodiments, each electrode set includes first and second elongated electrodes extending along a majority (e.g., at least one half, at least three quarter, at least five eighth) of the longitudinal dimension of the inflatable balloon, and the electrodes of each electrode set are configured to form an anode-cathode pair for bipolar delivery of radiofrequency ablative energy to target tissue. In some embodiments, the inflatable balloon may include compliant materials, and the balloon may be inflated to different diameters during treatment, for example, two different diameters at two different operating modes. In certain embodiments, the inflatable balloon can be inflated to a first diameter at a first operating mode, inflated to a second diameter at a second operating mode, inflated to a third diameter at a third operating mode, where the first diameter is different from the second diameter, the first diameter is different from the third diameter, and the second diameter is different from the third diameter. In some examples, the second diameter is greater than the first diameter and the third diameter is greater than the second diameter.

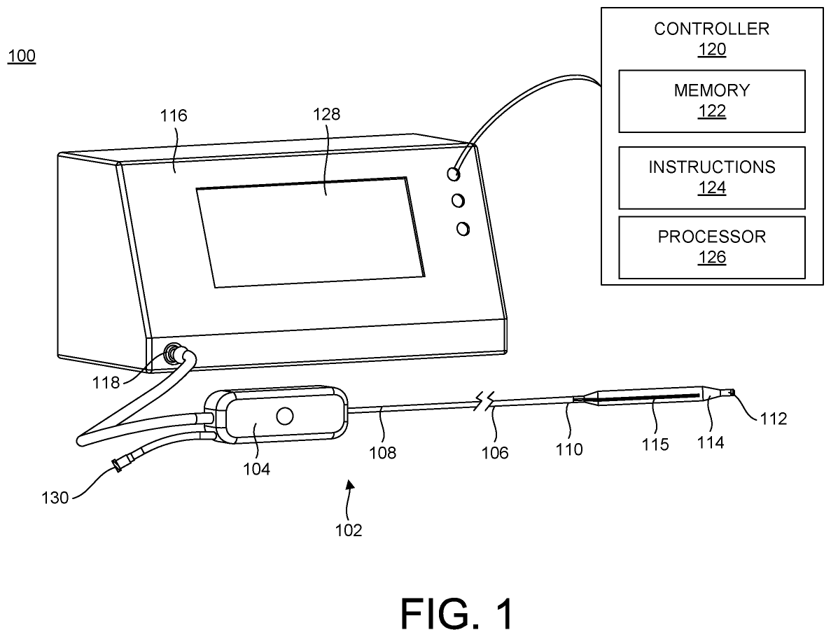

FIG. 1 is a schematic illustration of an exemplary ablation device 100 for treating chronic venous diseases, e.g., varicose veins, according to an embodiment of the present disclosure. The ablation device 100 includes an ablation catheter 102 including a handle 104, an elongated shaft 106 having a proximal end 108 and a distal end portion 110 terminating at a distal end 112, and a heating element 114 disposed near the distal end 112 of the elongated shaft 106. The shaft 106 is sized and configured such that the distal end 112 may be inserted into a target blood vessel. The heating element 114 is configured to deliver ablative energy (e.g., radiofrequency energy, thermal energy) to a wall of a target blood vessel.

The ablation device 100 may include an energy generator 116 electrically coupled to the handle 104 via a connector 118 and configured to generate energy by delivering an electric signal (e.g., currents, radiofrequency alternating currents). A controller 120 is operatively connected to the energy generator 116 to control the generation of the electric signal. The controller 120 can be implemented using firmware, integrated circuits, and/or software modules that interact with each other or are combined together. For example, the controller 120 may include memory 122 storing computer-readable instructions/code 124 for execution by a processor 126 (e.g., microprocessor) to perform aspects of embodiments of methods discussed herein.

According to certain embodiments, the heating element 114 employs structural features and/or components to improve the clinical performance as well as enhance the manufacturability of the ablation catheter 102. In some embodiments, as will be discussed in more details below, the heating element 114 may include an expandable component 115, also referred to as an inflatable balloon, having a proximal end and an opposite distal end and defining a longitudinal dimension (e.g., 3 centimeters, 7 centimeters) therebetween, and a plurality of electrode sets circumferentially spaced about the expandable component 115 and operatively coupled to the energy generator 116. In some embodiments, each electrode set includes first and second elongated electrodes extending along a majority of the longitudinal dimension of the expandable component 115, and the electrodes of each electrode set are configured to form an anode-cathode pair for bipolar delivery of radiofrequency ablative energy to target tissue. In certain examples, the first and second elongated electrodes have a same length. In some examples, a length of the first elongated electrode is greater than half of a length of the expandable component 115. In certain examples, a length of the first elongated electrode is greater than three fourth of a length of the expandable component 115.

According to some embodiments, the ablation device 100 includes a fluid source 130 fluidly connected to the expandable component 115. In certain embodiments, the expandable component 115 is deflated when the ablation device at a first state and inflated by fluid (e.g., saline, gas, etc.) from the fluid source 130 at a second state. In some embodiments, the expandable component 115 has an elongated shape, for example, the length of the expandable component 115 is at least two (2) times of the diameter of the expandable component 115. In some examples, the length of the expandable component 115 is at least three (3) times of the diameter of the expandable component 115.

In some embodiments, the controller 120 may be configured to communicate with various components of the device 100 and generate a graphical user interface (GUI) to be displayed via a display 128. The controller 120 may include any type of computing device suitable for implementing embodiments of the disclosure. Examples of computing devices include specialized computing devices or general-purpose computing devices such as workstations, servers, laptops, portable devices, desktop, tablet computers, hand-held devices, general-purpose graphics processing units (GPGPUs), and the like, all of which are contemplated within the scope of FIG. 1 with reference to various components of the device 100.

In some embodiments, the controller 120 includes a bus that, directly and/or indirectly, couples the following devices: a processor, a memory, an input/output (I/O) port, an I/O component, and a power supply. Any number of additional components, different components, and/or combinations of components may also be included in the computing device. The bus represents what may be one or more busses (such as, for example, an address bus, data bus, or combination thereof). Similarly, in some embodiments, the computing device may include a number of processors, a number of memory components, a number of I/O ports, a number of I/O components, and/or a number of power supplies. Additionally, any number of these components, or combinations thereof, may be distributed and/or duplicated across a number of computing devices.

In some embodiments, the memory 122 includes computer-readable media in the form of volatile and/or nonvolatile memory, transitory and/or non-transitory storage media and may be removable, nonremovable, or a combination thereof. Media examples include Random Access Memory (RAM); Read Only Memory (ROM); Electronically Erasable Programmable Read Only Memory (EEPROM); flash memory; optical or holographic media; magnetic cassettes, magnetic tape, magnetic disk storage or other magnetic storage devices; data transmissions; and/or any other medium that can be used to store information and can be accessed by a computing device such as, for example, quantum state memory, and/or the like. In some embodiments, the memory 122 stores computer-executable instructions for causing a processor (e.g., the controllers 120) to implement aspects of embodiments of system components discussed herein and/or to perform aspects of embodiments of methods and procedures discussed herein.

The computer-executable instruction 124 may include, for example, computer code, machine-useable instructions, and the like such as, for example, program components capable of being executed by one or more processors associated with a computing device. Program components may be programmed using any number of different programming environments, including various languages, development kits, frameworks, and/or the like. Some or all of the functionality contemplated herein may also, or alternatively, be implemented in hardware and/or firmware.

In some embodiments, the memory 122 may include a data repository implemented using any one of the configurations described below. A data repository may include random access memories, flat files, XML files, and/or one or more database management systems (DBMS) executing on one or more database servers or a data center. A database management system may be a relational (RDBMS), hierarchical (HDBMS), multidimensional (MDBMS), object oriented (ODBMS or OODBMS) or object relational (ORDBMS) database management system, and the like. The data repository may be, for example, a single relational database. In some cases, the data repository may include a plurality of databases that can exchange and aggregate data by data integration process or software application. In an exemplary embodiment, at least part of the data repository may be hosted in a cloud data center. In some cases, a data repository may be hosted on a single computer, a server, a storage device, a cloud server, or the like. In some other cases, a data repository may be hosted on a series of networked computers, servers, or devices. In some cases, a data repository may be hosted on tiers of data storage devices including local, regional, and central.

Various components of the device 100 can communicate via or be coupled to via a communication interface, for example, a wired or wireless interface. The communication interface includes, but not limited to, any wired or wireless short-range and long-range communication interfaces. The wired interface can use cables, umbilicals, and the like. The short-range communication interfaces may be, for example, local area network (LAN), interfaces conforming known communications standard, such as Bluetooth® standard, IEEE 702 standards (e.g., IEEE 702.11), a ZigBee® or similar specification, such as those based on the IEEE 702.15.4 standard, or other public or proprietary wireless protocol. The long-range communication interfaces may be, for example, wide area network (WAN), cellular network interfaces, satellite communication interfaces, etc. The communication interface may be either within a private computer network, such as intranet, or on a public computer network, such as the internet.

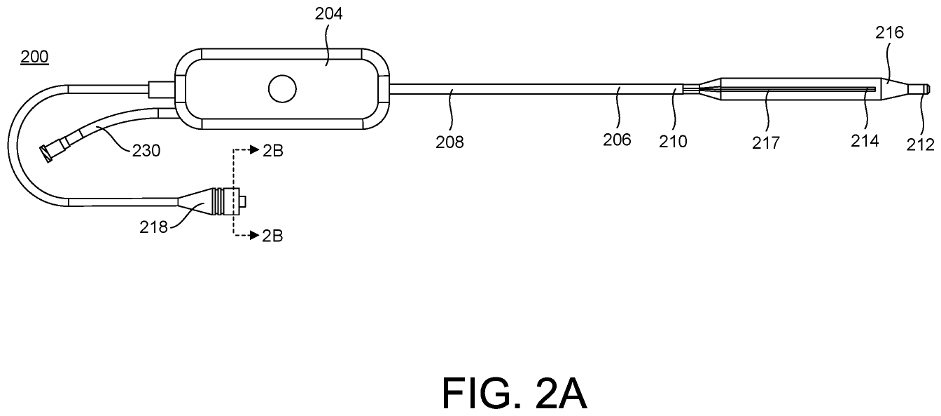

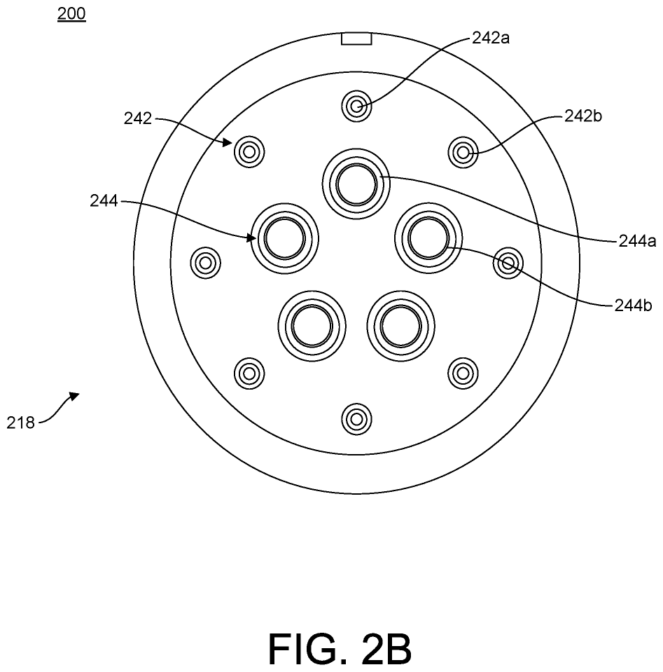

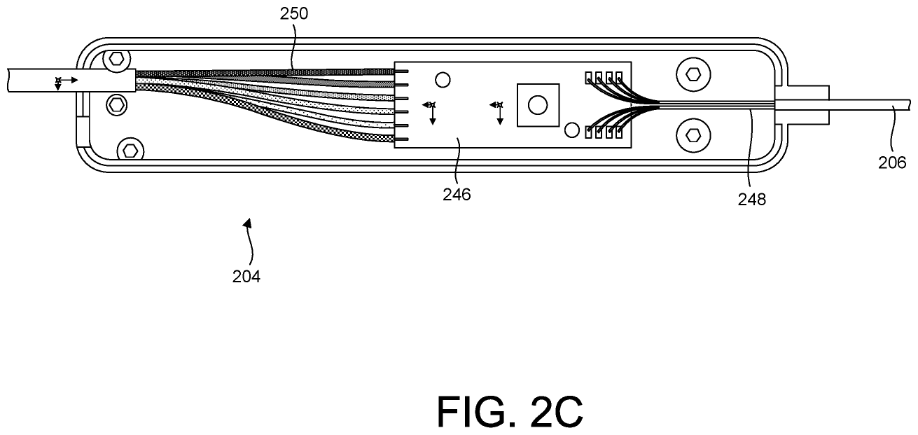

FIG. 2A is a schematic illustration of an exemplary ablation catheter 200 including a connector 218 (similar to the connector 118 as shown in FIG. 1) for treating chronic venous diseases, e.g., varicose veins; FIG. 2B is a schematic cross-sectional view of the connector 218 of the exemplary ablation catheter 200 along the cross-sectional indicator lines 2B-2B of FIG. 2A; FIG. 2C is a schematic cross-sectional view of the handle 204 of the exemplary ablation catheter of FIG. 2A, according to embodiments of the present disclosure.

As shown, the ablation catheter 200 includes a handle 204, an elongated shaft 206 having a proximal end 208 and a distal end portion 210 terminating at a distal end 212, and a heating element 214 disposed near the distal end 212 of the elongated shaft 206. The shaft 206 is sized and configured such that the distal end 212 may be inserted into a target blood vessel. The heating element 214 is configured to deliver ablative energy (e.g., radiofrequency energy, thermal energy) to the wall of a target blood vessel.

In some embodiments, as will be discussed in more details below, the heating element 214 may include an inflatable balloon 216 having a proximal end and an opposite distal end and defining a longitudinal dimension therebetween, and a plurality of electrode sets 217 circumferentially spaced about the balloon 216 and operatively coupled to an energy generator (e.g., the energy generator 116 in FIG. 1). In some embodiments, each electrode set 217 includes first and second elongated electrodes extending along a majority of the longitudinal dimension of the balloon, and the electrodes of each electrode set are configured to form an anode-cathode pair for bipolar delivery of radiofrequency ablative energy to target tissue. During treatment, the inflatable balloon 216 may be inflated and/or deflated via a fluid source 230. The fluid source 230 may be attached to a pump or syringe (not shown). In embodiments, the fluid source 230 may include a valve to prevent the inflatable balloon 216 from deflating during treatment. In some embodiments, for example as shown in FIG. 2A, the fluid source 230 may be connected to the inflatable balloon 216 via the handle 204 and elongated shaft 206. In some embodiments, the fluid source 230 may be directly connected to the inflatable balloon 216 (not shown).

In some embodiments, the connector 218 includes pins of different sizes 242 (including e.g., pins 242a, 242b) and 244 (including e.g., pins 244a, 244b). The pins 242 are relatively smaller than pins 244, and are configured to transfer electric signals (e.g., the electric signal generated by the energy generator 116 in FIG. 1). Exemplary electric signals may include thermocouple signals or pressure signals. The pins 244 are relatively larger compared to pins 242, and may be configured to allow current to pass from an energy generator (e.g., the energy generator 116 in FIG. 1) to generate heat on the heating element 214. One of the pins 244 may be used as a pin connected to ground (i.e., a ground pin). In some embodiments, where the heating elements include multiple heating segments (e.g., coil segments), the ground pin may be used as a common ground pin by the multiple heating segments.

As shown in FIG. 2C, electrode sets (e.g., electrode sets 217 as shown in FIG. 2A) may be connected to a printed circuit board (“PCB”) 246 located in the handle 204 via one or more wires 248 within the elongated shaft 206. In some embodiments, the one or more wires 248 may be copper wires. The PCB 246 may be connected to a generator (e.g., the energy generator 116 in FIG. 1) via one or more cables 250.

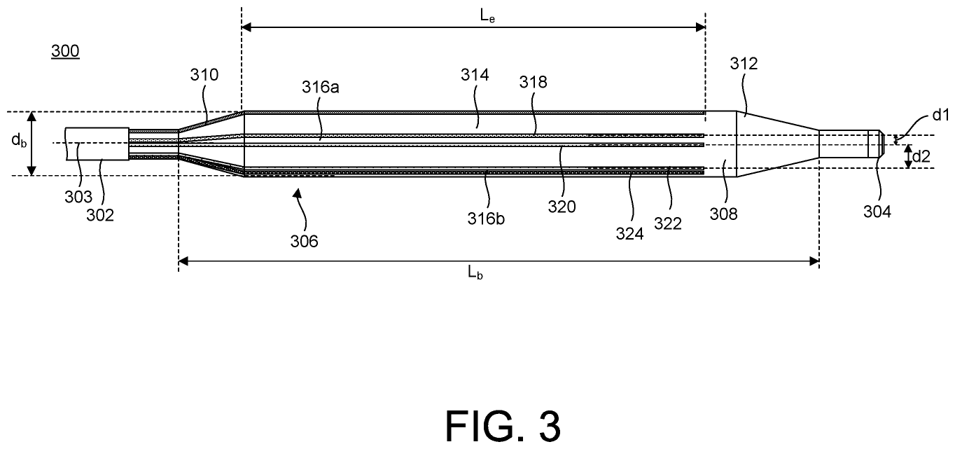

FIG. 3 is a schematic partial blown-up view of the distal end portion 300 of an ablation catheter in an expanded state, according to embodiments of the present disclosure. As shown, the distal end portion 300 includes part of an elongate shaft 302 terminating at a distal end 304 defining a longitudinal axis 303, and a heating element 306 disposed near the distal end 304 of the elongated shaft 302. The shaft 302 and the heating element 306 are sized and configured such that the distal end 304 may be inserted into a target blood vessel.

The heating element 306 may include an inflatable balloon 308 having a proximal end 310 and an opposite distal end 312 and defining a longitudinal dimension 314 therebetween, and a plurality of electrode sets 316 circumferentially spaced about the balloon 308 and operatively coupled to an energy generator (e.g., the energy generator 116 in FIG. 1). As veins may become tortuous due to chronic venous diseases, it is not easy for operators to insert the distal end portion 300 of an ablation catheter into the target vein. Placement of the heating element 306 on the distal end portion 300 to a specific treatment site may become increasingly difficult if the catheter is too stiff. Using an inflatable balloon 308 as a part of the heating element 306 may increase flexibility of the catheter, making it easier for the distal end portion 300 to go through tortuous veins and arrive at target treatment site, which may also reduce the operation time.

In some embodiments, each electrode set 316 includes first and second elongated electrodes (e.g., 318 and 320; or 322 and 324, as shown) extending along a majority of the longitudinal dimension 314 of the balloon 308, and the electrodes 318-324 of each electrode set 316 are configured to form an anode-cathode pair for bipolar delivery of radiofrequency ablative energy to target tissue. In the exemplary embodiments as shown in FIG. 3, the electrode 318 of the electrode set 316a is an anode that carries a positive charge, and the electrode 320 of the electrode set 316b is a cathode that carries a negative charge. Similarly, the electrode set 316b includes an anode electrode 322 and a cathode electrode 324. In some embodiments, at least one electrode in the plurality of electrode sets 316 includes a flexible circuit. In some embodiments, the electrode in the plurality of electrode sets 316 include flexible circuits.

The plurality of electrode sets 316 may be electroplated or metal sprayed, or produced using any method commonly used for producing flexible circuits as understood by a skilled artisan. In some instances, flexible circuits may be disposed onto the inflatable balloon 308 using adhesives. In some embodiments, the plurality of electrode sets 316 include materials similar to typical materials used for flexible circuits. In some embodiments, the plurality of electrode sets 316 include materials with relatively small electrical resistance.

In some embodiments, the distance d1 between the anode-cathode pair (i.e. distance between anode electrode 318 and cathode electrode 320) is smaller than a distance d2 between two adjacent electrode sets (i.e. distance between electrode set 316a and electrode set 316b as measured by the distance between the cathode electrode 320 and the anode electrode 322, distance between two adjacent electrode sets being the distance between two adjacent electrodes each in a respective electrode set). In some instances, the distance between two adjacent electrode is at least two (2) times of the distance between the anode-cathode pair. In embodiments, the distance d1 between each of the anode-cathode pair (i.e. distance between anode electrode 318 and cathode electrode 320; or the distance between anode electrode 322 and cathode electrode 324) may be the same. In certain examples, the first and second elongated electrodes have a same length Lₑ. In some examples, a length of the first elongated electrode Lₑ is greater than half of a length L_{b} of the inflatable balloon 308. In certain examples, a length of the first elongated electrode Lₑ is greater than three fourth of a length L_{b} of the inflatable balloon 308.

According to some embodiments, the inflatable balloon 308 is fluidly connected to a fluid source (e.g., the fluid source 130 in FIG. 1). In certain embodiments, the inflatable balloon 308 is deflated at a first state and inflated via a fluid source (e.g., by saline, gas, etc.) at a second state. In some embodiments, the inflatable balloon 308 has an elongated shape, for example, the length L_{b} of the inflatable balloon 308 is at least two (2) times of the diameter d_{b} of the inflatable balloon 308. In some examples, the length L_{b} of the inflatable balloon 308 is at least three (3) times of the diameter d_{b} of the inflatable balloon 308.

In some embodiments, the inflatable balloon 308 has a length L_{b} of from about three (3) centimeters to about ten (10) centimeters. In some embodiments, the inflatable balloon 308 has a diameter d_{b} of from about three (3) millimeters to about twelve (12) millimeters when inflated. In some embodiments, the inflatable balloon 308 has a diameter d_{b} of from about five (5) millimeters to about ten (10) millimeters when inflated. In some instances, the length L_{b} of the balloon 308 may be at least two times of the diameter d_{b} of the balloon 308 when inflated. In some embodiments, during treatment, the inflatable balloon 308 may have a diameter d_{b} greater than a diameter of a target vessel when inflated.

During treatment, the inflatable balloon 308 may be inflated to press against the target vein wall. A controller (e.g., the controller 120 in FIG. 1) may be configured to measure impedance between the electrode sets 316. Depending on a change of measured impedance, the controller may be configured to determine if the balloon 308 is contacting the target vessel wall without the need of an additional pressures sensor.

As mentioned above, the diameter of the varicose vein being treated may vary depending on the patient, or the location of treatment (e.g., the Greater Saphenous Vein may range in diameter from about 2.5 mm to about 14.0 mm at the femoral junction, from about 1.5 mm to about 12.0 mm in the thigh, and from about 1.0 mm to about 8.0 mm in the calf. The Lesser Saphenous Vein may range from about 1.5 mm to about 3.0 mm). Having the inflatable balloon 308 with adjustable width may help a physician adapt the same catheter for treatment of blood vessels with different diameters, or different sections within a certain vessel, and perfectly fit the blood vessel wall to achieve better therapeutic effect.

During treatment, the inflatable balloon 308 may be inflated to occlude the target vessel, which avoids blood flow through the vessel and increase thermal efficiency of the treatment. In embodiments, the balloon may be inflated to different sizes according to the diameter of the target vessel, such that one or more of the electrode sets 316 press against the wall of the target vessel. In some embodiments, a controller (e.g., the controller 120 in FIG. 1) may be configured to measure impedance between the electrode sets 316 to determine if the balloon 308 is contacting the vessel wall, thus enabling an operator to estimate the degree of ablation based on impedance information measured by the controller.

In some cases, the inflatable balloon 308 is inflated with a fluid. In some cases, the fluid is saline. In one example, the fluid is a gas. In one example, the fluid is nitrous oxide (N₂O). In one case, the inflatable balloon 308 is semi-compliant. In another case, the inflatable balloon 308 includes non-compliant material. If the balloon material is non-compliant, the distances from the electrodes to tissues can be known. If the balloon material is semi-compliant, the distances from the electrodes to tissues can be known, for example, with known pressure in the balloon.

In embodiment, the inflatable balloon 308 includes materials such as, for example, polyvinyl chloride (PVC), polyethylene (PE), cross-linked polyethylene, polyolefins, polyolefin copolymer (POC), polyethylene terephthalate (PET), nylon, polymer blends, polyester, polyimide, polyamides, polyurethane, silicone, polydimethylsiloxane (PDMS) and/or the like. The inflatable balloon 308 may include relatively inelastic polymers such as PE, POC, PET, polyimide or a nylon material. The membrane of the inflatable balloon 308 may be constructed of relatively compliant, elastomeric materials including, but not limited to, a silicone, latex, urethanes, or Mylar elastomers. The inflatable balloon 308 can be embedded with other materials such as, for example, metal, nylon fibers, and/or the like. The inflatable balloon 308 can be constructed of a thin, non-extensible polymer film such as, for example, polyester, flexible thermoplastic polymer film, thermosetting polymer film, and/or the like.

In embodiment, the membrane of the inflatable balloon 308 can be about 5-50 micrometers in thickness to provide sufficient burst strength and allow for foldability. In one embodiment, the membrane of the inflatable balloon 308 can have a thickness in the range of 25-250 micrometers. In one embodiment, the membrane of the inflatable balloon 308 can have tensile strength of 30,000-60,000 psi.

In one implementation, the balloon includes an insulative material. In some implementations, the electrodes 318-324 may include a thin film of an electro-conductive or optical ink. The ink can be polymer-based. The ink may additionally include materials such as carbon and/or graphite in combination with conductive materials. The electrode may include a biocompatible, low resistance metal such as silver, silver flake, gold, and platinum which are additionally radiopaque.

In some embodiments, the shaft 302 may be made of polyether ether ketone (“PEEK”), polycarbonate (“PC”), Pebax®, high density polyethylene (“HDPE”), polyimide (“PI”), or any suitable polymer material for manufacturing a catheter shaft as known to a skilled person in the art. In some embodiments, the inflatable balloon 308 may be made of Pebax®, polyethylene terephthalate (“PET”), thermoplastic polyurethane (“TPU”), nylon, polyamide (“PA” or “nylon plastic”) or any suitable polymer or synthetic thermoplastic polymer material as known to a skilled person in the art.

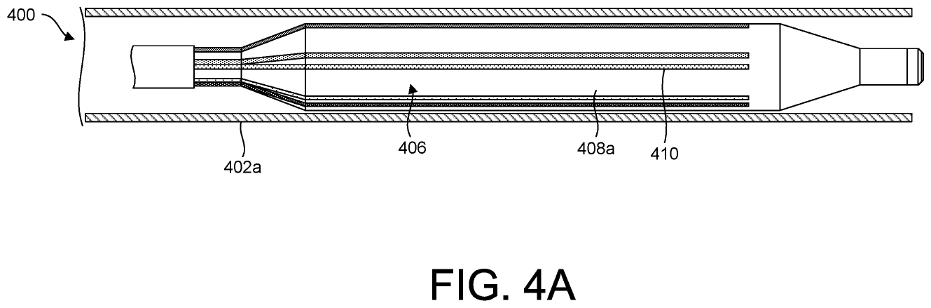

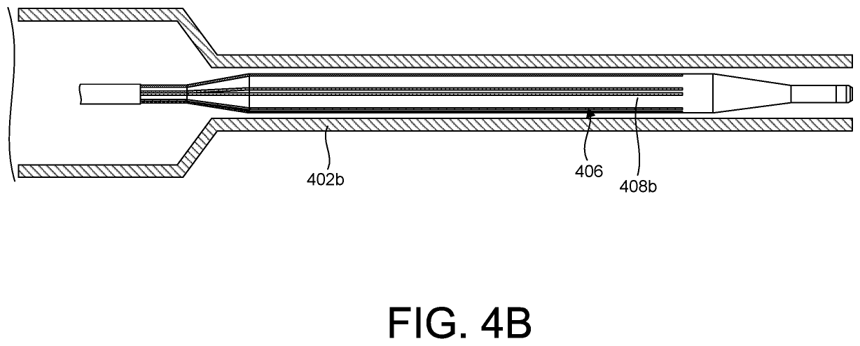

FIGS. 4A and 4B are schematic illustrations of a portion of an ablation catheter for use in a target blood vessel in a patient for treatment of varicose veins, according to embodiments of the present disclosure.

In some embodiments, during endovenous thermal ablation procedure, an introducer sheath may be positioned inside a patient's target vein using ultrasonic guidance and standard vascular technique. An ablation catheter (e.g., the ablation catheter 102 in FIG. 1) may then be inserted into the target vein through the introducer sheath. In some circumstances, under ultrasonic guidance, tumescent anesthetic solution or saline may be injected into target vein segment to act as a heat sink that protects tissue from thermal injury, and improve thermal conductivity between the wall of target vein and the ablation catheter.

As shown in FIG. 4A, the distal end portion 400 of an ablation catheter (e.g., the ablation catheter 102 in FIG. 1) is positioned in a target blood vessel 402a. The ablation catheter may be introduced and positioned with an introducer sheath using ultrasonic guidance. As will be appreciated by a skilled artisan, any standard vascular technique may be used here to introduce and position the distal end portion 400 of the ablation catheter into the target vein segment. The distal end portion 400 may include a heating element 406 having an inflatable balloon 408a and a plurality of electrode sets 410 circumferentially spaced about the balloon and operatively connected to an energy generator (e.g., the energy generator 116 in FIG. 1).

During treatment, the inflatable balloon 408a may be inflated (e.g., via the fluid source 130 in FIG. 1) to press against the target vein wall when the ablation catheter is at a first state (e.g., an expanded state), for example, as illustrated in FIG. 4A. A controller (e.g., the controller 120 in FIG. 1) may be configured to measure impedance between the electrode sets 410. The impedance may change before and after the inflatable balloon 408a contacts a target vein wall (e.g., the impedance may be large without contact, then decrease upon initial contact between the inflatable balloon 408a and a target vein wall, and then increase again as the treatment goes on). Depending on a change of measured impedance, the controller may be configured to determine if the balloon 408a at the first state is contacting the target vessel wall without the need of an additional pressures sensor. Having the inflatable balloon may help a physician adapt the same catheter for treatment of blood vessels with different diameters, and perfectly fit the blood vessel wall to achieve better therapeutic effect.

In some embodiments, during treatment, current may be applied to the plurality of electrode sets 410 by a generator (e.g., the energy generator 116 in FIG. 1). The generator may include a radiofrequency generator that generates radiofrequency current to heat the plurality of electrode sets 410. In some implementations, the ablation catheter may include a temperature sensor disposed along the length of a shaft of the catheter, and power delivery to the electrode sets 410 may be adjusted automatically by a controller (e.g., the controller 120 in FIG. 1) based on temperature or signals indicative of temperature measured by the temperature sensor. In some embodiments, a temperature sensor may be disposed along the length of the distal end portion 400. In some embodiments, a temperature sensor may be disposed on the inflatable balloon 408a and contacting one of the plurality of electrode sets 410. In some embodiments, one of the plurality of electrode sets 410 may be a thermocouple electrode set.

A segment of the target blood vessel 402a adjacent the plurality of electrode sets 410 being treated will close (e.g., shrink, reduced in diameter) as energy is delivered to the plurality of electrode sets 410, shown as 402b in FIG. 4B. External pressure may be applied as needed during treatment. After a certain section is treated (i.e. the section of the vein is closed), the catheter may be moved towards the venous access, and the process repeated until the entire vein is closed. The catheter and introducer sheath may then be removed, and the inflated balloon 408b may be deflated (e.g., via the fluid source 130 in FIG. 1) and then removed after treatment is done. In some use cases, a diameter of the heating element 406 and/or the balloon 408b is smaller than a diameter of blood vessel 402a and the heating element 406 can be moved close to the vessel wall during the treatment.

As the terms are used herein with respect to measurements (e.g., dimensions, characteristics, attributes, components, etc.), and ranges thereof, of tangible things (e.g., products, inventory, etc.) and/or intangible things (e.g., data, electronic representations of currency, accounts, information, portions of things (e.g., percentages, fractions), calculations, data models, dynamic system models, algorithms, parameters, etc.), “about” and “approximately” may be used, interchangeably, to refer to a measurement that includes the stated measurement and that also includes any measurements that are reasonably close to the stated measurement, but that may differ by a reasonably small amount such as will be understood, and readily ascertained, by individuals having ordinary skill in the relevant arts to be attributable to measurement error; differences in measurement and/or manufacturing equipment calibration; human error in reading and/or setting measurements; adjustments made to optimize performance and/or structural parameters in view of other measurements (e.g., measurements associated with other things); particular implementation scenarios; imprecise adjustment and/or manipulation of things, settings, and/or measurements by a person, a computing device, and/or a machine; system tolerances; control loops; machine-learning; foreseeable variations (e.g., statistically insignificant variations, chaotic variations, system and/or model instabilities, etc.); preferences; and/or the like.

Although illustrative methods may be represented by one or more drawings (e.g., flow diagrams, communication flows, etc.), the drawings should not be interpreted as implying any requirement of, or particular order among or between, various steps disclosed herein. However, certain some embodiments may require certain steps and/or certain orders between certain steps, as may be explicitly described herein and/or as may be understood from the nature of the steps themselves (e.g., the performance of some steps may depend on the outcome of a previous step). Additionally, a “set,” “subset,” or “group” of items (e.g., inputs, algorithms, data values, etc.) may include one or more items, and, similarly, a subset or subgroup of items may include one or more items. A “plurality” means more than one.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

1. A device for treating varicose vein, comprising:
a catheter comprising:
an elongated shaft having a proximal end and a distal end, the shaft being sized and configured such that the distal end can be inserted into a target blood vessel; and
a heating element disposed near the distal end of the elongated shaft, the heating element comprising:
an inflatable balloon having a proximal end and an opposite distal end and defining a longitudinal dimension therebetween; and
a plurality of electrode sets disposed circumferentially about the balloon, wherein each electrode set comprises first and second elongated electrodes extending along a majority of the longitudinal dimension of the balloon, wherein the electrodes of each electrode set are configured to form an anode-cathode pair for bipolar delivery of radiofrequency ablative energy to target tissue of the target blood vessels.

2. The device of claim 1, wherein the inflatable balloon has a length greater than three (3) centimeters.

3. The device of claim 2, wherein the inflatable balloon has a length smaller than ten (10) centimeters.

4. The device of claim 1, wherein the inflatable balloon has a diameter greater than five (5) millimeters when inflated.

5. The device of claim 1, wherein the inflatable balloon has a diameter greater than twelve (12) millimeters when inflated.

6. The device of claim 1, wherein the inflatable balloon has a diameter greater than a diameter of the target blood vessel when inflated.

7. The device of claim 1, wherein the inflatable balloon has a length and a diameter, wherein the length is at least two times of the diameter when inflated.

8. The device of claim 1, wherein at least one electrode in the plurality of electrode sets comprises a flexible circuit.

9. The device of claim 1, wherein a distance between an anode-cathode pair is smaller than a distance between two adjacent electrode sets.

10. The device of claim 9, the distance between two adjacent electrode sets is at least two (2) times of the distance between the anode-cathode pair.

11. A system for treating varicose vein, comprising:
a catheter comprising:
an elongated shaft having a proximal end and a distal end, the shaft being sized and configured such that the distal end can be inserted into a target blood vessel; and
a heating element disposed near the distal end of the elongated shaft, the heating element comprising:
an inflatable balloon having a proximal end and an opposite distal end and defining a longitudinal dimension therebetween; and
a plurality of electrode sets disposed circumferentially about the balloon, wherein each electrode set comprises first and second elongated electrodes extending along a majority of the longitudinal dimension of the balloon, wherein the electrodes of each electrode set are configured to form an anode-cathode pair for bipolar delivery of radiofrequency ablative energy to target tissue of the target blood vessels;
an energy generator connected to the catheter and configured to generate an electric signal; and
a controller operatively connected to the energy generator to control the generation of the electric signal.

12. The system of claim 11, wherein the plurality of electrode sets are operatively coupled to the energy generator.

13. The system of claim 11, wherein the inflatable balloon is configured to be inflated to a first diameter at a first operating mode and the inflatable balloon is configured to be inflated to a second diameter at a second operating mode, wherein the first diameter is different from the second diameter.

14. The system of claim 13, wherein the inflatable balloon is configured to be inflated to a size diameter such that an expandable membrane of the inflatable balloon is pressed against a wall of the target blood vessel.

15. The system of claim 14, wherein the controller is configured to received a measured impedance between the plurality of electrode sets to determine if the inflatable balloon is contacting the wall of the target blood vessel.

16. A catheter for treating varicose veins, the catheter comprising:
an elongated shaft having a proximal end and a distal end, the shaft being sized and configured such that the distal end can be inserted into a target blood vessel; and
an inflatable balloon disposed at the distal end of the shaft, the inflatable balloon having a proximal end and an opposite distal end and defining a longitudinal dimension therebetween, wherein the longitudinal dimension is at least two times a diameter of the balloon when inflated; and
a plurality of electrode sets disposed circumferentially about the balloon, wherein each electrode set comprises first and second elongated electrodes extending along a majority of the longitudinal dimension of the balloon, wherein the electrodes of each electrode set are configured to form an anode-cathode pair for bipolar delivery of radiofrequency ablative energy to target tissue of the target blood vessels.

17. The catheter of claim 16, wherein at least one electrode in the plurality of electrode sets comprises a flexible circuit.

18. The catheter of claim 17, wherein a distance between an anode-cathode pair is smaller than a distance between two adjacent electrode sets.

19. The catheter of claim 18, the distance between two adjacent electrode sets is at least two times of the distance between the anode-cathode pair.

20. The catheter of claim 16, wherein the inflatable balloon is configured to be inflated to a first diameter at a first operating mode and the inflatable balloon is configured to be inflated to a second diameter at a second operating mode, wherein the first diameter is different from the second diameter.
